(19) United States
(12) Patent Application Publication
(10) Pub. No.: US 2013/0345559 A1
(43) Pub. Date: Dec. 26, 2013

(54) QUANTITATIVE PERFUSION ANALYSIS FOR EMBOLOTHERAPY

(71) Applicant: MUSC FOUNDATION FOR RESEACH DEVELOPMENT, Charleston, SC (US)

(72) Inventors: Dieter HAEMMERICH, Charleston, SC (US); Matthew R. DREHER, Rockville, MD (US); Karun V. SHARMA, McLean, VA (US); Bradford J. WOOD, Bethesda, MD (US)

(73) Assignee: MUSC FOUNDATION FOR RESEACH DEVELOPMENT, Charleston, SC (US)

(21) Appl. No.: 13/852,897

(22) Filed: Mar. 28, 2013

Related U.S. Application Data
(60) Provisional application No. 61/616,926, filed on Mar. 28, 2012.

(51) Int. Cl. A61B 5/0275

(52) U.S. Cl. CPC A61B 5/0275
USPC 600/431

(57) ABSTRACT

Methods for quantitative perfusion analysis for embolotherapy are presented. The method quantitatively measures blood flow changes based on angiographic information. The method may provide potential evaluation of optimal embolization endpoints in vascular vessels. The method may be used in various applications such as transcatheter arterial chemoembolization (TACE), or other medical procedures that affect blow flow within bodily tissues. The method is applicable towards treatment of tumors in liver, kidney, brain, and other organs.

The present application claims the priority benefit of U.S. provisional application No. 61/616,926, filed Mar. 28, 2012, the entire contents of which are incorporated herein by reference.

### STATEMENT REGARDING FEDERALLY SPONSORED RESEARCH OR DEVELOPMENT

This invention was made with government support under Grant/Contract No. R01CA118990-01 A2 awarded by National Institute of Health. The government has certain rights in the invention.

### BACKGROUND OF THE INVENTION

1. Field of the Invention

This invention relates to quantitative perfusion analysis for embolotherapy and more particularly relates to quantitative measurement of blood flow changes in perfused tissues, such as tumors, liver parenchyma, kidney, prostate, and brain.

2. Description of the Related Art

Transcatheter arterial chemoembolization (TACE) is a minimally invasive treatment option for cancer patients and is clinically increasingly used in patients with unresectable hepatocellular carcinoma (HCC) and some hepatic metastases. This treatment is performed by selectively catheterizing tumor supplying arteries and identifying the abnormal angiographic tumor blush prior to delivery of chemotherapeutic drugs and embolic agents. TACE is performed using either drug eluting beads (DEBs) or a mixture of lipiodol/chemotherapy/embolic agents (conventional TACE). In either case, successful treatment requires adequate visualization and recognition of the tumor blush and delineation of this region from adjacent normal liver parenchyma prior to delivery of the therapy. During TACE, assessment of antegrade blood flow and reflux is essential for treatment monitoring and to determine the treatment endpoint.

Recently, several publications have reported on treatment of inoperable colorectal liver metastases (CRLM) refractory to systemic chemotherapy with conventional TACE and DEB-TACE with Iriniotecan (DEBIRI). Treatment of CRLM with TACE presents some additional challenges when compared to treatment of HCC. For example, CRLM are much less conspicuous on angiography with limited tumor blush. Even though multiple CRLM are often seen on pre-procedural contrast enhanced CT, MRI, or PET/CT, the same lesions are often difficult to visualize and delineate from adjacent parenchyma on angiography, even with the use of contemporary flat panel detector technology. In addition, because of the multifocal nature of CRLM and likely presence of micrometastases which are angiographically occult, catheter tip position tends to be less selective or lobar in order to target diffuse disease. These factors also make determination of embolization endpoint (i.e. the degree of blood flow reduction or stasis for DEB-TACE) or lipiodol deposition (for conventional TACE) more difficult to assess in cases of CRLM compared to HCC.

Techniques have been employed to aid angiographic detection of vascular abnormalities such as color coded images and/or arrival time maps that may also provide a subjective assessment of blood flow. Currently, the optimal embolization endpoint is unknown and determined by the operators' experience based on subjective and qualitative visual assessment of blood flow reduction in the embolized arteries seen on angiography during the procedure. Therefore, there is a need for quantitative measurement of blood flow changes that occur in targeted tumors and liver parenchyma during TACE.

### SUMMARY OF THE INVENTION

Embodiments of methods for quantitative perfusion analysis for embolotherapy are presented. The method quantitatively measures blood flow changes based on angiographic information. The method may provide potential evaluation of optimal embolization endpoints in vascular vessels. The method may be used in various applications such as transcatheter arterial chemoembolization (TACE), hepatocellular carcinoma (HCC), or the like.

In one embodiment, the method comprises injecting a contrast agent into one or more vascular vessels, obtaining a set of angiography images, where the set of angiography images in a time series and associated with flow of the contrast agent in the one or more vascular vessels, calculating a time of arrival (TOA) of the contrast agent at each of one or more selected locations associated the one or more vascular vessels, and presenting the TOA to a user. The calculation of TOA of the contrast agent is based on the flow of the contrast agent, which is recorded by the set of angiography images. The vascular vessel may be feeding vessels of a tumor, or vessels of liver parenchyma or vessels in other organs and tissues.

In one embodiment, the set of angiography images are two-dimensional (2-D) images. In an alternative embodiment, the set of angiography images are three-dimensional (3-D) images. In one embodiment, a subset of angiography images are selected from the set of angiography images, and the contrast agent is visible in each of the subset of angiography images.

In one embodiment, calculating a TOA of the contrast agent at a selected location on a vascular vessel comprises calculating a temporal slope at the selected location at each of a subset of angiography images, and determining the TOA as the time at which the angiography image with maximum temporal slope is obtained. The temporal slope at a location of an angiography image is defined as dI/dt, the derivative of the image intensity Ito time t.

In another embodiment, calculating a TOA of the contrast agent at a selected location on a vessel comprises determining a reference signal; correlating the reference signal with the intensity time course at selected locations in the set of angiography images; and determining the TOA as the time at which maximum correlation between the reference signal and the selected locations is obtained. The region of interest is selected to be inside the vascular vessel and distal from an outlet of a contrast agent injecting device, such as a catheter.

In certain embodiments, the method may comprise identifying one or more vascular regions in each of the set of angiography images. In order identify the vascular regions, maximum intensity projection (MIP) may be performed on the set of angiography images to generate an MIP image, and a region growing algorithm on the MIP images to identify connected vascular regions.

In some embodiments, the method may comprise compensating motions of the vascular vessels in the set of angiography images. The motion of a vascular vessel may be caused by, e.g. the catheter contact the vascular vessel, or beating of organs associated with the vascular vessel, or the like. Vessel motion compensation may comprise defining and applying a mask to each of the set of angiography images. The mask may be the MIP image generated by maximum intensity projection.

In one embodiment, an affine transformation may be performed on the set of angiography images based on a reference angiography image. The reference angiography image may be an image in which the contrast agent is visible in the one or more vascular vessels. The parameters of the affine transformation are selected such that deviation between the set of angiography images and the reference angiography image is minimzed. The deviation between the set of angiography images and the reference angiography image may be evaluated as sum of squared differences between pixel intensities, sum of absolute differences between pixel intensities, or the like.

In one embodiment, the method may further comprise calculating a flow velocity of the contrast agent at a selected location within a vascular vessel, where the calculating is based on the TOA, e.g. a distance between two selected locations in the vessel divided by the difference between TOA at the selected locations. The method may also comprise calculating a flow rate of the contrast agent, where the flow rate is calculated as the product of the flow velocity and a cross-sectional area of a vascular vessel.

In one embodiment, presenting the TOA comprises visually displaying the TOA at each of one or more selected locations in a TOA map.

In one embodiment, TOAs of contrast agent before the vascular vessel is embolized are compared to TOAs of contrast agent after the vascular vessel is embolized. The result may be visually compared in images.

As used herein the specification, “a” or “an” may mean one or more. As used herein in the claim(s), when used in conjunction with the word “comprising”, the words “a” or “an” may mean one or more than one.

“Open” means free from lateral constraint by solid objects except for constraint by a single supporting surface that provides lateral constraint in one direction.

The word “particle” includes any substance, including an inorganic material, liquid droplet, molecule such as DNA, RNA or other subcellular components.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 is a flow chart illustrating one embodiment of a method for quantitative perfusion analysis.

FIG. 2 is a flow chart illustrating one embodiment of a method for quantitative perfusion analysis.

FIG. 3 is a flow chart illustrating one embodiment of a method for quantitative perfusion analysis.

FIG. 4 is a flow chart illustrating one embodiment of a method for quantitative perfusion analysis.

FIGS. 5A-5C shows an angiography image series for quantitative perfusion analysis.

FIG. 6 illustrates vessel segmentation for quantitative perfusion analysis.

FIG. 7 illustrates blood flow analysis using time of arrival (TOA) of contrast agent.

FIG. 8 compares two TOA estimating methods.

FIG. 9 illustrates a diagram where TOA map of contrast agent is overlaid on a digital subtraction angiography (DSA) image.

FIG. 10 illustrates quantitative perfusion analysis results for an hepatocellular carcinoma (HCC) example.

FIG. 11 illustrates quantitative perfusion analysis results for a colorectal cancer (CRC) example.

FIG. 12 illustrates quantitative perfusion analysis results for another CRC example.

FIG. 13 illustrates calculation of flow velocity for quantitative perfusion analysis.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept will become apparent to those having ordinary skill in the art from this disclosure.

The flow chart diagrams that follow are generally set forth as logical flow chart diagrams. As such, the depicted order and labeled steps are indicative of one embodiment of the present disclosure. Other steps and methods may be employed that are equivalent in function, logic, or effect to one or more steps, or portions thereof, of the illustrated method. Additionally, the format and symbols employed are provided to explain logical steps and should be understood as not limiting the scope of an invention. Although various arrow types and line types may be employed in the flow chart diagrams, they should be understood as not limiting the scope of the corresponding method. Indeed, some arrows or other connectors may be used to indicate only the logical flow of the method. For instance, an arrow may indicate a waiting or monitoring period of unspecified duration between enumerated steps. Additionally, the order in which a particular method occurs may or may not strictly adhere to the order of the corresponding steps shown.

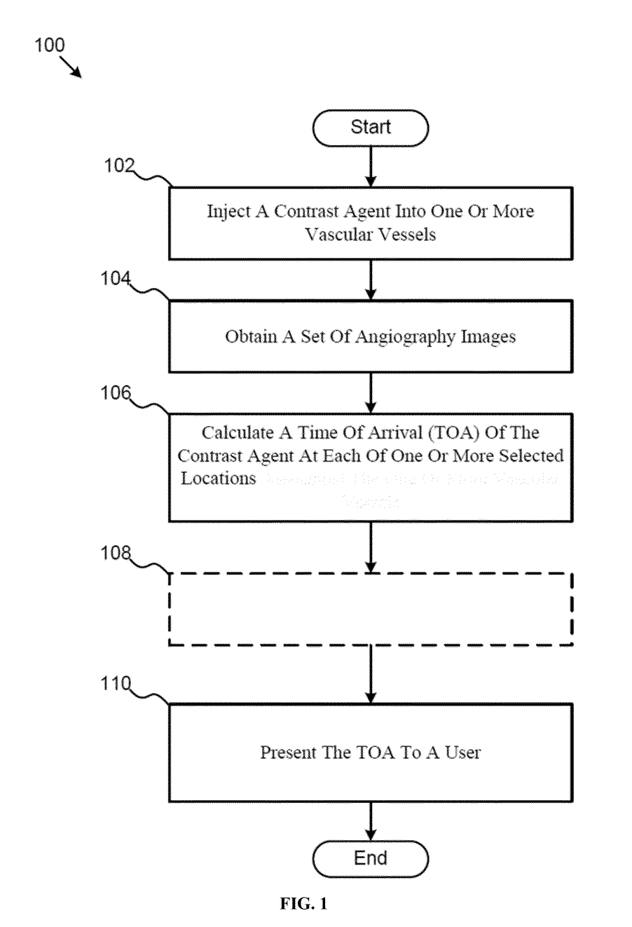

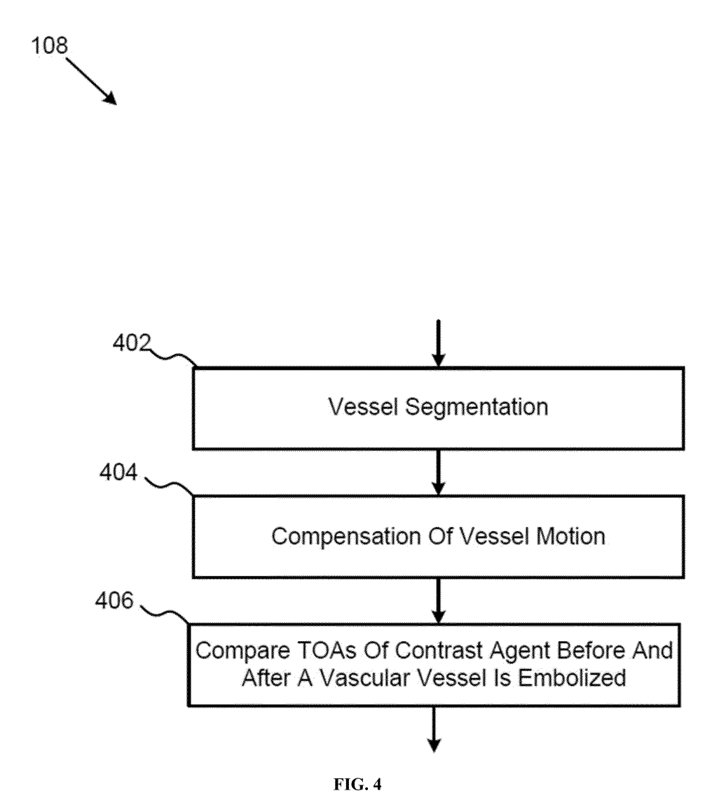

FIG. 1 illustrates one embodiment of a method 100 for quantitative perfusion analysis. The method may include injecting a contrast agent into one or more vascular vessels; obtaining a set of angiography images, the set of angiography images in a time series and associated with flow of the contrast agent in the one or more vascular vessels; calculating a time of arrival (TOA) of the contrast agent at each of one or more selected locations associated the one or more vascular vessels; and presenting the TOA to a user. The method 100 may also include optional steps 108 as described further in FIG. 4. Further, the method may be applied to locations in tissues where no vasculature is visible to determine TOA within microvasculature of these tissue locations.

The image data may be obtained by the method of fluoroscopy (also known as digital subtraction angiography). Contrast agent can be injected into tumor feeding vessels by an intravascular catheter. A set of 2D angiography images comprising a time series may be acquired at, e.g. a rate of 2 frames/second. The time series thus visualized the filling of large tumor vessels, tumor microvasculature, and tissue with contrast agent.

The set of angiography images may be used to calculate TOA of the contrast agent at a selected location on the vessels. Vessel segmentation may be performed on the angiography images to identify the vessel regions. Vessel segmentation may be optional in certain embodiments of the analysis method. The TOA may be calculated based on the flow of the contract agent which is captured by the set of angiography images. The TOA of contrast agent may also illustrate the blood flow analysis of embolization, where the reduction of flow is analyzed, e.g. TOA is reduced after embolization due to slowing down of blood flow. The result may be visualized displayed in an image.

All image processing algorithms may be implemented via signal processing methods known by a person of ordinary skill in the art. Custom algorithms may be designed to analyze pre- and post-embolization angiographic image sequences with the goal of quantifying the contrast time of arrival (TOA) at different points along the embolized vessels, in the targeted tumor, and surrounding liver parenchyma.

TOA for each spatial location (e.g., pixel or voxel) may be determined from raw x-ray angiography data sets via two algorithms: (1) defined by time of maximum slope of contrast increase at each spatial location, or (2) by cross-correlation of time course of average contrast within a user-defined region near catheter tip (i.e. input function) with time course of contrast at each spatial location.

Maximum Slope

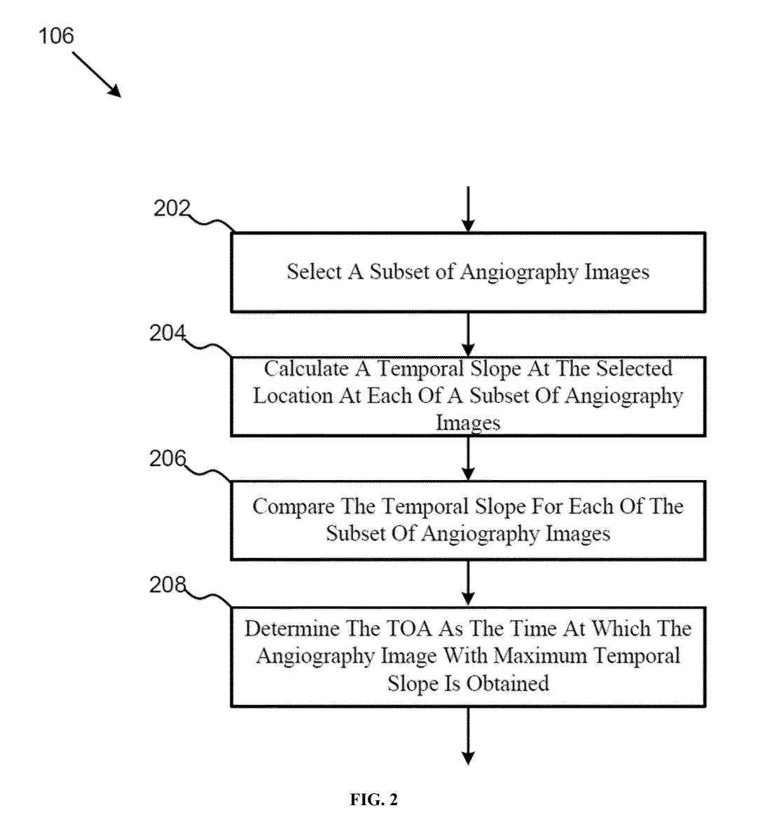

FIG. 2 illustrates one embodiment of a method for calculating TOA.

A subset of the image data time series was selected such that arrival of the contrast throughout the tumor or tissue region of interest is visible, while removing any data before contrast injection, and removing contrast washout phase data. At each pixel in the DSA imaging data, the temporal slope (dI/dt, where I is image intensity) was calculated throughout the time series, with subsequent Gaussian filtering along time (filter size=1 sample). Then, for each pixel the time where dI/dt is maximum was calculated. Time was referenced to the time where contrast arrived (dI/dt=max) at a defined reference location (this reference location is constant, if multiple analyses from different image data series for the same tumor are calculated). This allows for direct comparison in TOA between different datasets (e.g. before and after embolization).

TOA maps may be constructed and then visualized as color maps, with optional combination with the vessel mask to only show intravascular regions if desired.

Cross Correlation

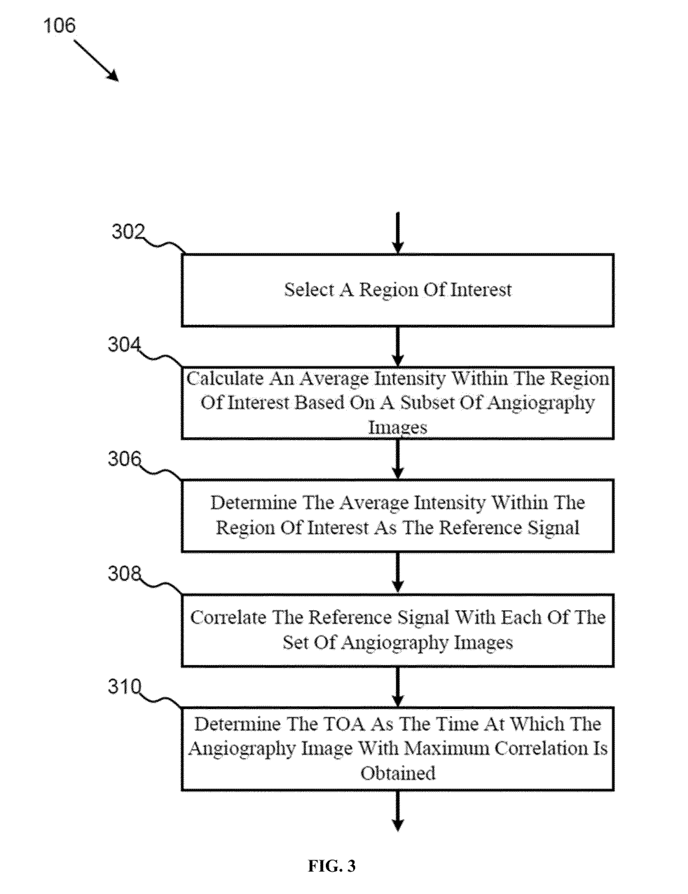

FIG. 3 illustrates another embodiment of a method for calculating TOA.

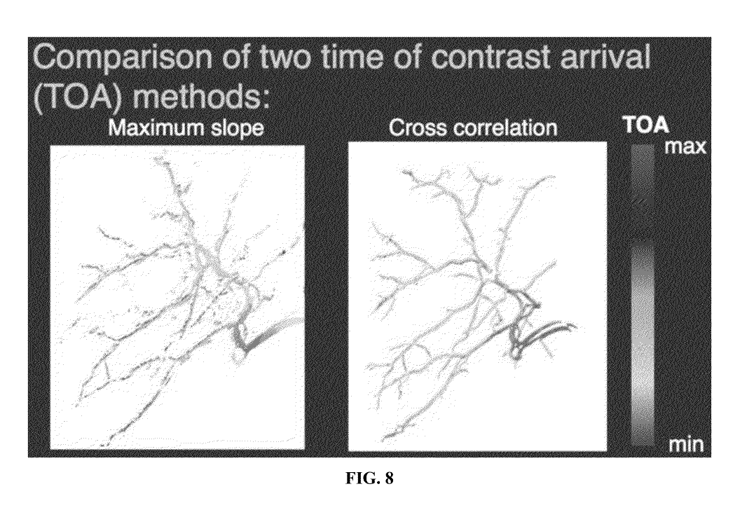

An input function may be defined as average intensity within a small region of interest, preferably circular, manually drawn or placed slightly distal from the intravascular catheter tip. This input function represents the change in contrast as the contrast agent is injected (e.g. FIG. 8, right image). Ideally, the injection rate and time are optimized such that a unique input function (e.g. pulse, or sequence of pulses) results, as this provides better results for the following correlation.

The input function is then cross-correlated with the intensity time course of every pixel in the image, or alternatively only of pixels defined by the vessel mask to reduce computation time. The correlation is highest at the point in time, when the match between input function and time course of contrast at a certain location is best, and indicates TOA of the contrast at a particular location (e.g., pixel) in the image. One potential advantage of using the cross correlation function is that it can be less sensitive to noise and motion artifacts compared to the Maximum Slope method. Visualization is similar as described in Maximum Slope method.

FIG. 4 illustrates optional steps 108 of method 100, as described in further detail below.

Vessel Segmentation

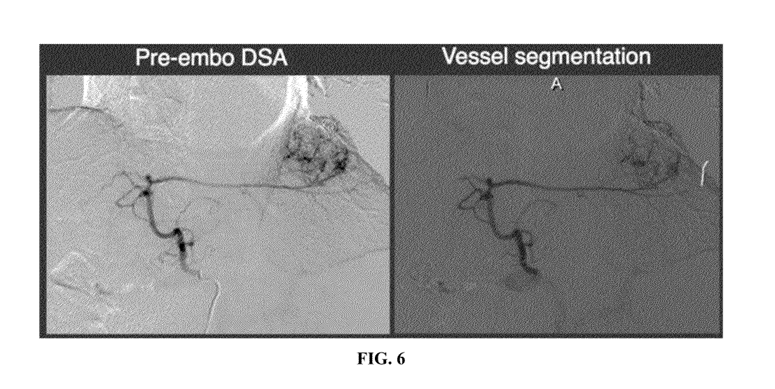

To identify intravascular regions, vessel segmentation was performed on an image created by maximum intensity projection (MIP) performed along the time axis throughout a digital subtraction angiography (DSA) image data set for each pixel. Within this MIP image, a region growing algorithm based on user-defined seed points placed inside vessels was performed to identify vascular regions, as shown in FIG. 6. A variety of other segmentation algorithms familiar to a person of ordinary skill in the art could be used alternatively to region growing.

Motion Compensation

Vascular vessel motions may be present due to, e.g. the catheter contacting the vessel, or organ movement (e.g., liver, heart, or the like) associated to the vessel, patient movement, or other effects. TOA analysis typically does not work well without motion compensation in data where there is motion, since it assumes that the location represented by a pixel throughout the time series is constant. Following vessel segmentation, motion compensation of the segmented vessel regions is performed. A vessel mask from the MIP image above may be defined where the vascular regions identified during vessel segmentation are enlarged by, e.g. 10-20 pixels. This mask may be applied to a DSA image series, and motion compensation is performed based on the masked image data set. Affine geometrical transformation may be performed between a masked image in the DSA time series and a masked reference image (manually selected where contrast was visible throughout the tumor vasculature) with transformation parameters such that deviation between each image and the reference image is minimal (using sum of squared differences between pixel intensities as cost function).

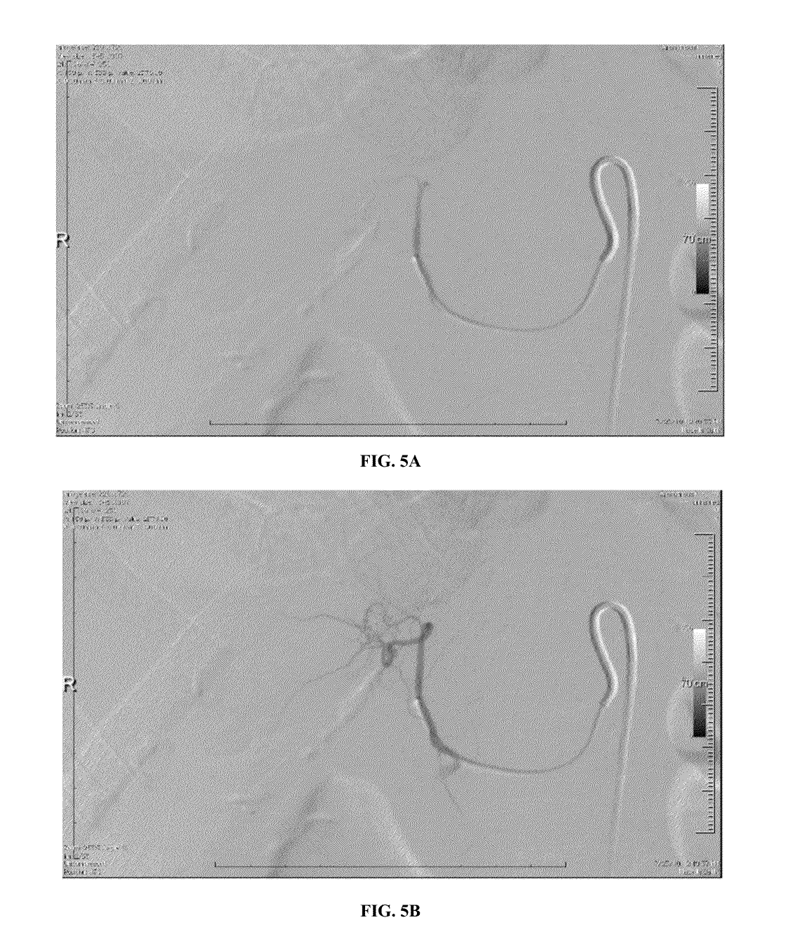

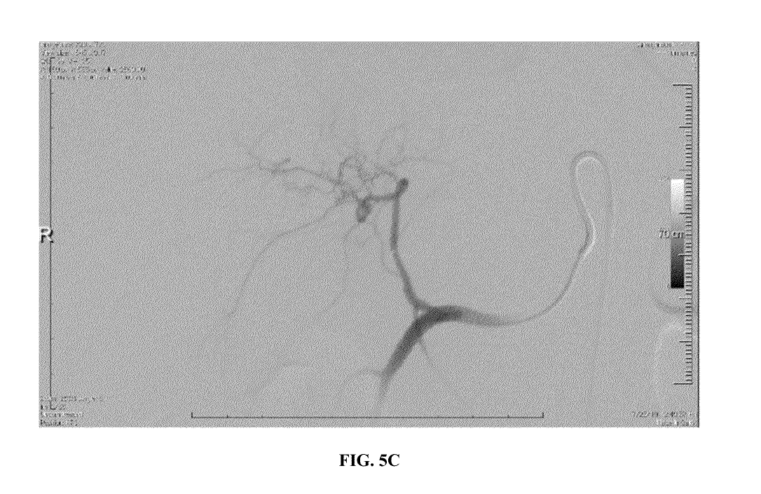

FIGS. 5A-5C shows an angiography image time series representative of image data that can be used for quantitative perfusion analysis. The images series show contrast agent flows from a catheter located approximately in the center through the vascular vessel network.

FIG. 6 shows results for vessel segmentation, where the left part of FIG. 6 shows a pre-embo DSA image obtained before embolization depicting a hypervascular tumor (upper right of image), and the right part of FIG. 6 shows interactive vessel segmentation accurately depicting the tumor feeding arterial supply vessels.

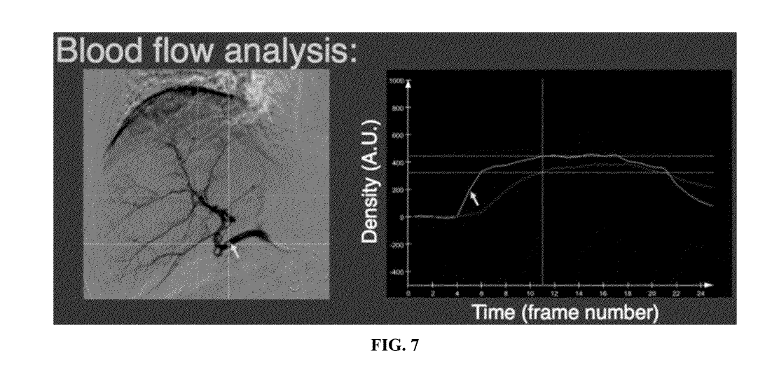

FIG. 7 illustrates blood flow analysis using TOA, where the left part of FIG. 7 shows a DSA image, and right part shows the time course of contrast agent at two locations indicated by the arrows in the left part of FIG. 7. The arrow in the right part of FIG. 7 indicates the time where slope (dI/dt, where I is the image intensity) is maximum, which corresponds with time of arrival (TOA).

FIG. 8 compares the TOA of contrast agent estimated by two methods: the maximum slope method and the cross correlation method.

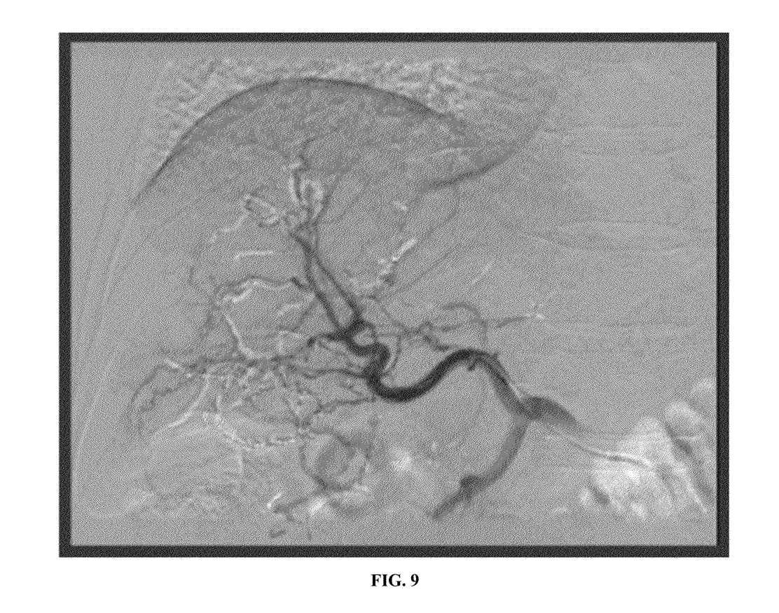

FIG. 9 illustrates a TOA map, where the TOAs of contrast agent at different locations is displayed, and the TOA map is overlaid on a digital subtraction angiography (DSA) image.

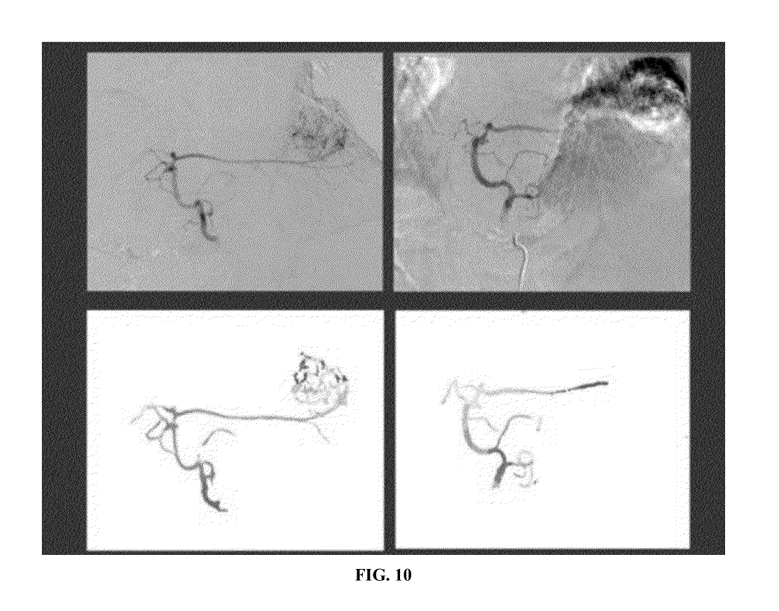

FIG. 10 illustrates quantitative perfusion analysis results for an example data set from a patient with primary liver tumor. Upper images show DSA images before (left), and after (right) embolization; lower images show TOA map before (left), and after (right) embolization.

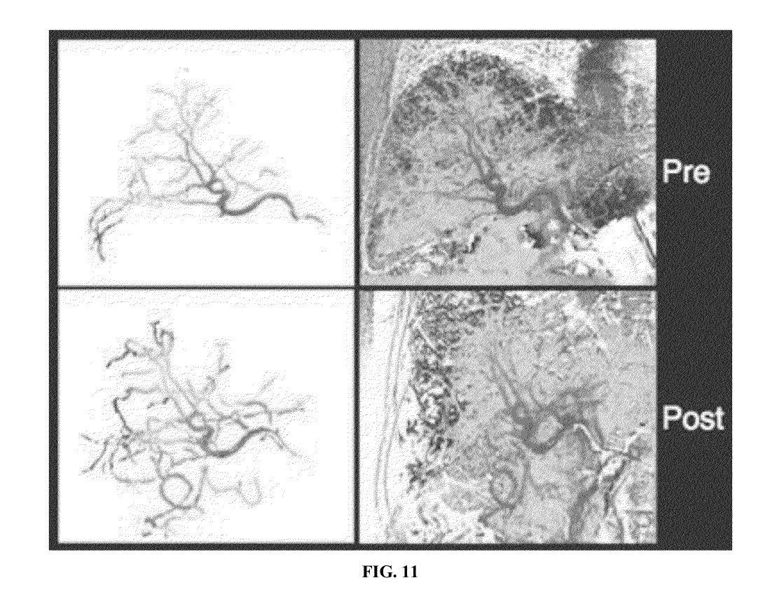

FIG. 11 illustrates quantitative perfusion analysis results for an example data set from a patient with liver metastases. It is noted that there is significant reduction in parenchymal flow as can be seen in the non-segmented images on the right.

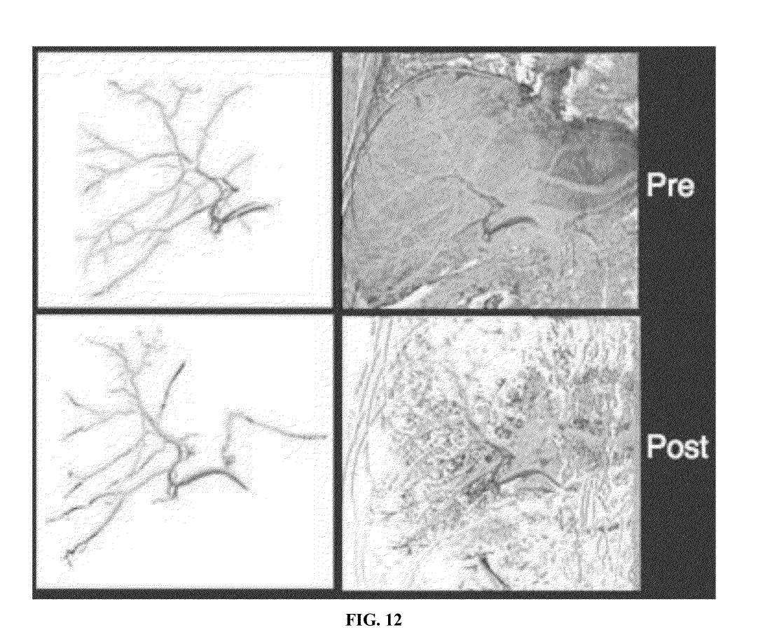

FIG. 12 illustrates quantitative perfusion analysis results for another colorectal cancer (CRC) example. In this example, the object is a 59 year-old man with hepatic dominant CRC metastases diagnosis in 2009, treated with FOLFOX and Avastin but disease progression noted, and treated with DEBIRI August 2010-September 2010. FIG. 9 shows the pre- and post-DEB-TACE blood flow changes. It is noted that there is significant reduction in parenchymal flow as can be seen in the non-segmented images on the right.

In the examples, primary liver cancer showed successive slowing of contrast arrival in tumor feeding arteries and delayed appearance of contrast in tumor after embolization. Similar changes in blood flow were found with liver metastases except that changes in tumor vasculature were not always apparent. In addition to feeding artery flow changes, regional or geographic perfusion changes were much more apparent in metastases and highlight the method's utility for identifying target and non-target embolization.

Flow Velocity and Flow Calculation

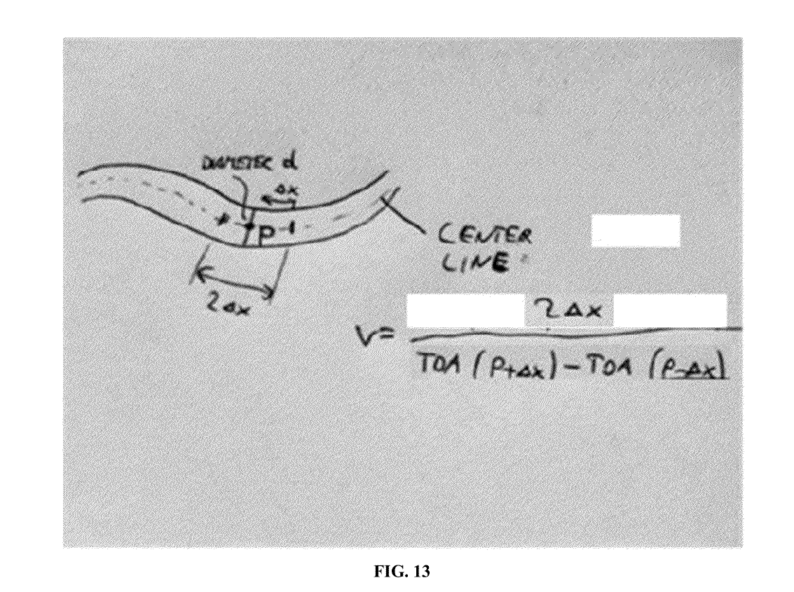

In additional steps, flow velocity and flow along vessels can be calculated from TOA values, as shown in FIG. 13. This requires segmentation of vessels as described above. From segmented vessel regions, the centerline, as well as diameter are identified along the vessels. Vessel cross sectional area is calculated from diameter. The flow velocity can then be calculated as shown below.

The volume flow rate may be calculated as the product of the flow velocity and cross-sectional area of the vessel.

Note that flow rate and flow velocities calculated above are subject to errors since calculation is based on a 2-D projection of vessel geometry. To correct for this error, 3-D angiography data sets are required. These 3-D data sets would have to be acquired in a separate imaging step before or after the perfusion analysis described below (acquired e.g. by rotational C-arm imaging systems), Correction of flow and flow velocity data then requires (1) spatial registration of 2-D fluoroscopy time series and 3-D data sets, and (2) projection of TOA data from 2-D onto the known 3-D geometry, and (3) calculation of flow and flow velocity based on dimensions derived from 3-D rather than 2-D data as described in paragraph above.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope of the invention. In addition, modifications may be made to the disclosed apparatus and components may be eliminated or substituted for the components described herein where the same or similar results would be achieved. All such similar substitutes and modifications apparent to those skilled in the art are deemed to be within the spirit, scope, and concept of the invention as defined by the appended claims.

What is claimed is:

1. A method comprising:
injecting a contrast agent into one or more vascular vessels;
obtaining a set of angiography images, the set of angiography images in a time series and associated with flow of the contrast agent in the one or more vascular vessels;
calculating a time of arrival (TOA) of the contrast agent at each of one or more selected locations associated the one or more vascular vessels; and
presenting the TOA to a user.

2. The method of claim 1, wherein the one or more vascular vessels are feeding vessels of a tumor.

3. The method of claim 1, configured to be operable in transcatheter arterial chemoembolization (TACE).

4. The method of claim 1, wherein calculating a TOA of the contrast agent at a selected location on a vascular vessel comprises:
calculating a temporal slope at the selected location at each of a subset of angiography images;
comparing the temporal slope for each of the subset of angiography images; and
determining the TOA as the time at which the angiography image with maximum temporal slope is obtained.

5. The method of claim 4, wherein the subset of angiography images are selected from the set of angiography images obtained in claim 1.

6. The method of claim 1, wherein calculating a TOA of the contrast agent at a selected location on a vessel comprises:
determining a reference signal;
determining a correlation of the reference signal with each of the set of angiography images; and
determining the TOA as the time at which the angiography image with a maximum correlation is obtained.

7. The method of claim 6, wherein determining a reference signal comprises:
selecting a region of interest; and
calculating an average intensity within the region of interest based on a subset of angiography images; and
determining the average intensity within the region of interest as the reference signal.

8. The method of claim 7, wherein the subset of angiography images are selected from the set of angiography images obtained in claim 6.

9. The method of claim 7, wherein the region of interest for determining the reference signal is inside the vascular vessel and distal from an outlet of a contrast agent injecting device.

10. The method of claim 1, further comprising identifying one or more vascular regions in each of the set of angiography images.

11. The method of claim 10, wherein identifying one or more vascular regions comprises performing maximum intensity projection (MIP) on the set of angiography images to generate an MIP image and performing a segmentation algorithm on the MIP images.

12. The method of 11, wherein the segmentation algorithm is a region growing algorithm.

13. The method of claim 1, further comprising compensating motions of the vascular vessels in the set of angiography images.

14. The method of claim 13, wherein compensating motions of the vascular vessels comprises defining and applying a mask to each of the set of angiography images and performing an affine transformation to the set of angiography images based on a reference angiography image.

15. The method of claim 14, wherein the reference angiography image is an image in which the contrast agent is visible in the one or more vascular vessels.

16. The method of claim 14, wherein parameters of the affine transformation are selected such that deviation between the set of angiography images and the reference angiography image is minimized.

17. The method of claim 16, wherein the deviation between the set of angiography images and the reference angiography image is evaluated as a sum of squared differences between pixel intensities.

18. The method of claim 1, wherein presenting the TOA comprises visually displaying the TOA at each of one or more selected locations in a TOA map.

19. The method of claim 1, further comprising comparing a TOA of contrast agent before the vascular vessel is embolized and a TOA of contrast agent after the vascular vessel is embolized.

20. The method of claim 1, further comprising calculating a flow velocity of the contrast agent at a selected location within a vascular vessel, the calculating based on the TOA.

21. The method of claim 20, wherein calculating a flow velocity comprises correcting errors resulting from use of 2-D image data by additional use of 3-D data.

22. The method of claim 20, further comprising calculating a flow rate of the contrast agent, the calculating based on the flow velocity and a cross-sectional area of a vascular vessel.

23. The method of claim 22, wherein calculating a flow rate comprises correcting errors resulting from use of 2-D image data by additional use of 3-D data.

24. The method of claim 1, wherein the set of angiography images are two-dimensional images.

25. The method of claim 1, wherein the set of angiography images are three-dimensional images.

26. A non-transitory computer-readable medium comprising a set of instructions executable by one or more processors, the set of instructions comprising:
obtaining a set of angiography images in a time series, the set of angiography images associated with a flow of a contrast agent in one or more vascular vessels;
calculating a time of arrival (TOA) of the contrast agent at each of one or more selected locations of the one or more vascular vessels, the calculating of time based on the flow of the contrast agent;
presenting the TOA to a user;
calculating the flow velocity or flow rate; and
presenting the flow velocity or flow rate to a user.
